(19) United States
(12) Patent Application Publication
(10) Pub. No.: US 2004/0161449 A1
(43) Pub. Date: Aug. 19, 2004

(54) Formulation and method for reduction of stress in meat-producing animals

(76) Inventor: Don L. Smothers, Dallas, TX (US)

Correspondence Address: JACQUELINE S LARSON, P O BOX 2426, SANTA CLARA, CA, 95055-2426, US

(21) Appl. No.: 10/774,712

(22) Filed: Feb. 9, 2004

Related U.S. Application Data
(63) Continuation-in-part of application No. PCT/US02/25136, filed on Aug. 8, 2002.
(60) Provisional application No. 60/310,956, filed on Aug. 8, 2001.

(51) Int. Cl. A23K 1/165; A61K 35/78

(52) U.S. Cl. 424/442; 424/730

(57) ABSTRACT

The present invention is directed to formulas comprising naturally-occurring ingredients and to treatment methods, for the reduction of stress in meat-producing animals.

This application is a continuation-in-part of International Application No. PCT/US02/025136, filed Aug. 8, 2002 and designating the United States, which claims benefit of U.S. Provisional application Ser. No. 60/310,956, filed Aug. 8, 2001, the disclosures of all of which are incorporated herein by reference.

### FIELD OF THE INVENTION

The present invention is directed to formulas and methods for the reduction of stress in meat-producing animals; for example, the stress associated with the movement of meat-producing animals from the production facility to the point of slaughter in the processing facility.

### BACKGROUND OF THE INVENTION

In the process of moving meat-producing animals from the production facility to the packing plant, animals are removed from the holding area (coop, pen, barnyard, etc.), moved down a narrow alleyway, and driven through a loading chute onto a livestock trailer. Animals are then transported for a variable number of hours to a packing facility where they are unloaded from the livestock trailer. The meat-producing animals then are moved to a scale, weighed, often tattooed or branded, and moved to a holding area (coop, pen, barnyard, etc.). After a rest period of a variable number of hours, the meat-producing animals then are moved into the slaughter area. This process of moving meat-producing animals usually is quite stressful to them. Many stressed meat-producing animals walk slowly, thus getting in the way of other of the animals, while some of the stressed animals exhibit extreme responses, resulting in their inability to move or even in death.

Moreover, the sensory qualities of the meat from meat-producing animals that were stressed prior to slaughter are not as high as they would otherwise have been, all other factors being equal. Typically, this is because more of the animal's muscle glycogen is broken down to glucose than with an unstressed animal. Subsequent metabolism of the glucose results in the formation of lactic acid. Lactic acid formation causes the pH of the muscle cell to drop. In living animals, the lactic acid is metabolized, thus causing the pH to return to normal, higher levels. If the excess lactic acid is not removed from the muscle cells, for example because the animal has been slaughtered, or if the bloodstream is not able to handle the excess lactic acid, a generalized acidosis may occur. If severe, acidosis may lead to death of a living animal.

In animals that already are dead (whether of natural causes, stress-induced reactions, or humane slaughter), an excess of lactic acid, if not removed from the cells, will cause the muscle pH to drop, thus causing the meat to taste and look less desirable. The industry-wide average pH of slaughtered meat-producing animals is approximately 5.75 after 24 hours (“ultimate pH”). The pH of animals that have experienced a stress reaction shortly before death is typically in the range of 5.3-5.4 or even lower. Many researchers have attempted to increase the pH of animal meat by various methods, and some have used epinephrine.

It is well known in the art that hormones from the adrenal gland can have a strong influence on animal metabolism and homeostasis. Adrenal hormones help animals to cope with stress through a series of reactions that increase circulation and help provide energy to the muscles. Epinephrine is one of the major adrenal hormones. The natural release of epinephrine readies the animal for stressful situations. Epinephrine binds to the muscle cell, and stimulates the breakdown of glycogen into energy-rich compounds, allowing the animal's normal metabolism to dissipate the lactic acid before the animal is slaughtered. However, if this elevated production of hormones from the adrenal gland occurs within approximately 72-96 hours of slaughter, there is not sufficient time for the lactic acid to dissipate and the pH of the animal's meat to return to a level for acceptable meat quality.

No one previously has been able to acclimate a meat-producing animal headed for slaughter in an easy, economical, and efficient manner such that the group of animals so acclimated includes fewer slow-walkers or prematurely dead animals, and the animals from a group that have been so acclimated yield meat that is consistently of high quality in terms of taste and appearance. Accordingly, there has been a long-felt need for a solution to this problem. No one has yet developed a commercially viable solution that can be used by farmers and slaughterers. Moreover, the U.S. federal Food and Drug Administration has not yet approved the regular use of epinephrine or epinephrine-like drugs in meat-producing animals to reduce stress.

An additional benefit of acclimating the meat-producing animals prior to the stress-inducing event is that often stress causes livestock (such as pigs, cattle, and poultry, for example) to shed salmonella. By avoiding the stress reaction, the meat produced by the claimed method will be less contaminated with salmonella.

### SUMMARY OF THE INVENTION

This invention is directed to a method of reducing the stress in a meat-producing animal and of improving the quality of the meat produced from slaughtering said animal. More particularly, the method comprises administering to the meat-producing animal an effective amount of a formulation of stress-reducing nutrients. The formulation is preferably administered to the animal for a period starting from about 72 to about 96 hours prior to a stressful event and up to the event.

The nutrient formulation of the invention comprises the following ingredients:

| | wt % |
|---|---|
| Ingredient | range (approx.) |
| Gamma Aminobutyric Acid (GABA) | 0-15 |
| Kava Kava Root | 0-60 |
| St. Johns Wort (0.3% Hypericum) | 0-20 |
| Damiana 4/1 | 7-40 |
| Valarian Root | 5-70 |
| Lithium Arginate | 0-5 |
| Niacinamide | 0.1-5 |
| Inositol | 0.1-3 |
| 5-Hydroxytryptophan | 0-5 |
| Passion Flower | 0-50 |
| Corydalis | 0-30 |
| Jujube | 0-20 |
| Skullcap | 0-20 |
| Glycine | 0-40 |

Additional ingredients that may be included in the formulation of the invention include, but are not limited to, suma, goto kola, lemon balm, hops, ginger, taurine, nicotinamide, and magnesium citrate.

### DETAILED DESCRIPTION OF THE INVENTION

The method and formulation of the present invention may be useful with any meat-producing animal. The method is particularly useful with meat-producing animals from the group that includes sheep, lambs, cattle, veal, turkeys, chickens, and most especially, swine.

In a presently preferred embodiment, the nutrient formulation of the invention comprises the following ingredients:

wt %

| Ingredient | range (approx.) | preferred (approx.) |
|---|---|---|
| Gamma Aminobutyric | 3-15 | 7 |
| Acid (GABA) | | |
| Kava Kava Root | 5-60 | 30 |
| St. Johns Wort | 3-20 | 10 |
| (0.3% Hypericum) | | |
| Damiana 4/1 | 7-40 | 15 |
| Valarian Root | 5-70 | 33 |
| Lithium Arginate | 0.1-5 | 2 |
| Niacinamide | 0.1-5 | 2 |
| Inositol | 0.1-3 | 1 |

In a second presently preferred embodiment, the nutrient formulation of the invention comprises the following ingredients:

wt %

| Ingredient | range (approx.) | preferred (approx.) |
|---|---|---|
| Damiana 4/1 | 7-40 | 18 |
| Valarian Root | 5-70 | 42 |
| Niacinamide | 0.1-5 | 1 |
| Inositol | 0.1-3 | 1 |
| 5-Hydroxytryptophan | 0.1-5 | 2 |
| Passion Flower | 5-50 | 22 |
| Glycine | 4-40 | 14 |

In a third presently preferred embodiment, the nutrient formulation of the invention comprises the following ingredients:

| Ingredient | wt % (range, approx.) |
|---|---|
| Damiana 4/1 | 15-22 |
| Valarian Root | 25-50 |
| 5-Hydroxytryptophan | 1-5 |
| Passion Flower | 15-39 |
| Glycine | 5-20 |

In a fourth presently preferred embodiment, the nutrient formulation of the invention comprises the following ingredients:

| Ingredient | wt % (range, approx.) |
|---|---|
| St. John's Wort | 8-15 |
| Damiana 4/1 | 15-25 |
| Valarian Root | 25-40 |
| Passion Flower | 5-20 |
| Corydalis | 5-20 |
| Jujube | 1-15 |
| Skullcap | 1-15 |

In preparing the formulation of the invention, the ingredients are mixed or blended together to obtain a homogeneous mixture. If desired, a wet granulation may be prepared, wherein the homogeneous mixture is wetted to a slurry, dried and ground to the desired particle size.

This invention further involves the administration of an effective amount of the nutrient formulation to the meat-producing animals. Administration is preferably begun about 72 to about 96 hours prior to a potentially stressful event such as, for example, the process of loading the animals to travel to the slaughterhouse or the period of time the animals are in the slaughterhouse. Administration is normally continued up until the stressful event occurs. The formulation is conveniently administered by adding it to or mixing it into the normal feed ration given to the animals. An effective amount of the formulation is added to the feed; by “effective amount” is meant an amount that is effective in eliminating elevated secretions from the adrenal glands of the animal when the animal is exposed to the stressful event. The particular amount will be dependent on the type of meat-producing animal being treated, as well as its age, weight and sex. The particular amount can be determined by normal procedures known to those of skill in the art without undue experimentation.

Using swine as an example, it has been found that when swine are fed the nutrient formulation of the invention in their normal feed immediately prior to a stress-inducing event, the quality of the meat from the treated animals upon slaughter is demonstrably higher than that from animals not fed the nutrient formulation.

The formulation of the invention is made up of naturally-occurring ingredients and thus has significant desirable benefits. There are no residuals of synthetic drug in the resulting muscle tissue. Because of the small amounts of the formulation administered to the animal, the ingredients should not be present in or otherwise affect the meat. Even if there are minute amounts in tissue, they will not have any more effect than residuals from the animals' regular ration.

Kava Kava consists of rhizomes of Piper Methysticum G. Forster and contains Kava-pyrones. According to the Complete German Commission E Monographs, there are no known side effects. The herb has been safely used by humans for centuries.

St. John's Wort (Hypericum perforatum L.) has no known contraindications or interactions (Complete German Commission E Monographs); it is noted that there is a potential side effect of photo sensitization in fair-skinned individuals.

Damiana (Tumera diffusa) has no known risks (Complete German Commission E Monographs).

Valerian root (Valerianae officinalis L.) has no contraindications, side effects or interactions with other products (Complete German Commission E Monographs).

Suma (Pfaffia paniculata) is an herb grown and used throughout South America. It has been used for many years in South America, North America and Europe by humans with no reported negative side effects.

The present formulation and method have been tested with market-weight pigs, in a non-laboratory setting and at dosages from about 1.5 g to 15.0 grams total per animal, with outstanding success. The meat from the treated pigs has been of an extremely high quality and has muscle fibers of a satisfactory pH.

What is claimed is:

1. A stress-reducing formulation for treating a meat-producing animal, the formulation comprising:
0 wt % to about 15 wt % gamma aminobutyric acid (GABA),
0 wt % to about 60 wt % kava kava root,
0 wt % to about 20 wt % St. Johns Wort (0.3% hypericum),
about 7 wt % to about 40 wt % damiana 4/1,
about 5 wt % to about 70 wt % valerian root,
0 wt % to about 5 wt % lithium arginate,
about 0.1 wt % to about 5 wt % niacinamide,
about 0.1 wt % to about 3 wt % inositol,
0 wt % to about 5 wt % 5-hydroxytryptophan,
0 wt % to about 50 wt % passion flower,
0 wt % to about 30 wt % corydalis,
0 wt % to about 20 wt % jujube,
0 wt % to about 20 wt % skullcap, and
0 wt % to about 40 wt % glycine.

2. A formulation according to claim 1 comprising:
about 3 wt % to about 15 wt % gamma aminobutyric acid (GABA),
about 5 wt % to about 60 wt % kava kava root,
about 3 wt % to about 20 wt % St. Johns Wort (0.3% hypericum),
about 7 wt % to about 40 wt % damiana 4/1,
about 5 wt % to about 70 wt % valerian root,
about 0.1 wt % to about 5 wt % lithium arginate,
about 0.1 wt % to about 5 wt % niacinamide, and
about 0.1 wt % to about 3 wt % inositol.

3. A formulation according to claim 1 comprising:
about 7 wt % to about 40 wt % damiana 4/1,
about 5 wt % to about 70 wt % valerian root,
about 0.1 wt % to about 5 wt % niacinamide,
about 0.1 wt % to about 3 wt % inositol,
about 0.1 wt % to about 5 wt % 5-hydroxytryptophan,
about 5 wt % to about 50 wt % passion flower, and
about 4 wt % to about 40 wt % glycine.

4. A formulation according to claim 1 comprising:
about 15 wt % to about 22 wt % damania,
about 25 wt % to about 50 wt % valarian,
about 15 wt % to about 39 wt % passion flower,
about 1 wt % to about 5 wt % 5-hydroxytryptophan, and
about 5 wt % to about 20 wt % glycine.

5. A formulation according to claim 1 comprising:
about 8 wt % to about 15 wt % St. Johns wort,
about 15 wt % to about 25 wt % damania,
about 25 wt % to about 40 wt % valarian,
about 5 wt % to about 20 wt % corydalis,
about 1 wt % to about 15 wt % jujube, and
about 1 wt % to about 15 wt % skullcap.

6. A formulation according to claim 1 which further comprises at least one ingredient selected from the group consisting of suma, goto kola, lemon balm, hops, ginger, taurine, nicotinamide, and magnesium citrate.

7. A formulation according to claim 1 wherein the meat-producing animal is swine.

8. A method for reducing the stress in a meat-producing animal, the method comprising administering to the meat-producing animal an effective amount of the stress-reducing formulation of claim 1 prior to a stressful event.

9. A method according to claim 8 wherein the stress-reducing formulation is administered to the animal starting from about 72 to about 96 hours prior to the stressful event.

10. A method according to claim 9 wherein the stress-reducing formulation is administered to the animal up to the stressful event.

11. A method according to claim 8 wherein the stress-reducing formulation comprises:
about 3 wt % to about 15 wt % gamma aminobutyric acid (GABA),
about 5 wt % to about 60 wt % kava kava root,
about 3 wt % to about 20 wt % St. Johns Wort (0.3% hypericum),
about 7 wt % to about 40 wt % damiana 4/1,
about 5 wt % to about 70 wt % valerian root,
about 0.1 wt % to about 5 wt % lithium arginate,
about 0.1 wt % to about 5 wt % niacinamide, and
about 0.1 wt % to about 3 wt % inositol.

12. A method according to claim 8 wherein the stress-reducing formulation comprises:
about 7 wt % to about 40 wt % damiana 4/1,
about 5 wt % to about 70 wt % valerian root,
about 0.1 wt % to about 5 wt % niacinamide,
about 0.1 wt % to about 3 wt % inositol,
about 0.1 wt % to about 5 wt % 5-hydroxytryptophan,
about 5 wt % to about 50 wt % passion flower, and
about 4 wt % to about 40 wt % glycine.

13. A method according to claim 8 wherein the stress-reducing formulation comprises:
about 15 wt % to about 22 wt % damania,
about 25 wt % to about 50 wt % valarian,
about 15 wt % to about 39 wt % passion flower,
about 1 wt % to about 5 wt % 5-hydroxytryptophan, and
about 5 wt % to about 20 wt % glycine.

14. A method according to claim 8 wherein the stress-reducing formulation comprises:
about 8 wt % to about 15 wt % St. Johns wort,
about 15 wt % to about 25 wt % damania,
about 25 wt % to about 40 wt % valarian,
about 5 wt % to about 20 wt % corydalis,
about 1 wt % to about 15 wt % jujube, and
about 1 wt % to about 15 wt % skullcap.

15. A method according to claim 8 wherein the stress-reducing formulation further comprises at least one ingredient selected from the group consisting of suma, goto kola, lemon balm, hops, ginger, taurine, nicotinamide, and magnesium citrate.

16. A method according to claim 8 wherein the meat-producing animal is swine.

17. Use of a formulation to reduce the stress in a meat-producing animal, the stress-reducing formulation comprising:
0 wt % to about 15 wt % gamma aminobutyric acid (GABA),
0 wt % to about 60 wt % kava kava root,
0 wt % to about 20 wt % St. Johns Wort (0.3% hypericum),
about 7 wt % to about 40 wt % damiana 4/1,
about 5 wt % to about 70 wt % valerian root,
0 wt % to about 5 wt % lithium arginate,
about 0.1 wt % to about 5 wt % niacinamide,
about 0.1 wt % to about 3 wt % inositol,
0 wt % to about 5 wt % 5-hydroxytryptophan,
0 wt % to about 50 wt % passion flower,
0 wt % to about 30 wt % corydalis,
0 wt % to about 20 wt % jujube,
0 wt % to about 20 wt % skullcap, and
0 wt % to about 40 wt % glycine.

18. A use according to claim 17 wherein the stress-reducing formulation further comprises at least one ingredient selected from the group consisting of suma, goto kola, lemon balm, hops, ginger, taurine, nicotinamide, and magnesium citrate.

19. A use according to claim 17 wherein the meat-producing animal is swine.
